(19) United States
(12) Patent Application Publication
(10) Pub. No.: US 2025/0352497 A1
(43) Pub. Date: Nov. 20, 2025

(54) BIGUANIDE BASE COMPOSITIONS, BIGUANIDE BASE PHARMACEUTICAL COMPOSITIONS, AND THEIR PREPARATIONS AND USES

(71) Applicant: ADA FORSYTH INSTITUTE, INC., Cambridge, MA (US)

(72) Inventors: Jirun Sun, Cambridge, MA (US); Xuesong He, Cambridge, MA (US); Wenyuan Shi, Cambridge, MA (US)

(73) Assignee: ADA FORSYTH INSTITUTE, INC., Cambridge, MA (US)

(21) Appl. No.: 19/048,774

(22) Filed: Feb. 7, 2025

Related U.S. Application Data
(63) Continuation of application No. PCT/US2023/029897, filed on Aug. 9, 2023.
(60) Provisional application No. 63/396,574, filed on Aug. 9, 2022.

(51) Int. Cl. A61K 31/155; A61K 9/00; A61P 1/02

(52) U.S. Cl. CPC A61K 31/155; A61K 9/0063; A61P 1/02

(57) ABSTRACT

Disclosed herein are embodiments of biguanide (e.g., CHX) base compositions comprising one or more carriers and a biguanide (e.g., CHX) majorly in its base form (e.g., CHX base), pharmaceutical compositions thereof, and uses thereof. In certain embodiments, the biguanide base compositions disclosed herein showed unexpectedly preferred growth inhibition of acid-producing bacteria when compared with treatment of one or more salts of the same biguanide base.

### CROSS-REFERENCE TO RELATED APPLICATIONS

This application is a continuation of International Patent Application No. PCT/US2023/029897, filed Aug. 9, 2023, which claims the benefit of U.S. Provisional Patent Application No. 63/396,574, filed Aug. 9, 2022, which are incorporated herein by reference in their entirety.

### STATEMENT OF GOVERNMENT INTEREST

This invention was made with government support under grant nos. R01DE029479A and 7R21DE029925-02, awarded by the National Institutes of Health. The government has certain rights in the invention.

### TECHNICAL FIELD

This invention relates to biguanide (e.g., chlorhexidine (CHX)) base compositions, biguanide (e.g., CHX) base pharmaceutical compositions, and their preparation and uses.

### BACKGROUND

Chlorhexidine (CHX) is a biguanide base (CHX base), which has a low water solubility. Therefore, chlorhexidine salts (e.g., chlorhexidine gluconate and chlorhexidine digluconate (CHX-G) or chlorhexidine acetate, chlorhexidine chloride, etc.), which are more soluble than the CHX base, are widely used as disinfectants and antiseptic owing to the cationic chlorhexidine ions in water that break the cell membrane and achieve the antimicrobial efficacy.

### SUMMARY

Disclosed herein are embodiments of biguanide (e.g., CHX) base compositions comprising one or more carriers and a biguanide (e.g., CHX) majorly in its base form (e.g., CHX base). In certain embodiments, the biguanide (e.g., CHX) base composition showed unexpectedly preferred growth inhibition of acid-producing bacteria when compared with treatment of one or more salts of the same biguanide (e.g., CHX) base. Selective growth inhibition of acid-producing bacteria may avoid biocorrosion caused by acid produced by acidogenic bacteria while keeping cell viability of beneficial microbiome, e.g., probiotics. In certain embodiments, the carriers are solid and/or liquid. In certain embodiments, the biguanide (e.g., CHX) base interacts with the one or more carriers through physical interactions, chemical interactions, or a combination of both.

Disclosed are biguanide (e.g., CHX) base pharmaceutical compositions comprising biguanide (e.g., CHX) base and one or more pharmaceutically acceptable carriers.

Disclosed herein are methods for preparation of these biguanide (e.g., CHX) base compositions and biguanide (e.g., CHX) base pharmaceutical compositions.

Disclosed herein are methods for maintaining pH in a microbiome at about pH 5.5 or higher, the microbiome comprising acid-producing bacteria and non-acid-producing bacteria. In certain embodiments of the method disclosed herein, growth inhibitions of one or more of the acid-producing bacteria are more significantly than those of one or more non-acid-producing bacteria. In certain embodiments of the method disclosed herein, the pH of the microbiome was maintained at about 5.5 or higher without eliminating all or substantially all microbiome.

### BRIEF DESCRIPTION OF THE DRAWINGS

FIG. 1A shows release of CHX from CHX loaded MSN3 under different pH, the CHX loaded MSN3 was prepared according to Example I.

FIG. 1B compares release of CHX from CHX loaded MSN3 and release of CPC from CHX loaded MSN4 when pH was switched between pH 8 (shaded) and pH 4 (clear), the CHX loaded MSN3 and CPC loaded MSN4 were prepared according to Example I.

FIG. 2A shows the pH of biofilm prepared according to Example III treated with CHX base in solutions (CHX-50, 50 μg/mL; CHX-100, 100 μg/mL); CHX base loaded MSN (MSN3) at various concentrations (MSN3-25, 25 μg/mL; MSN3-50, 50 μg/mL; MSN3-75, 75 μg/mL; MSN3-100, 100 μg/mL; or MSN3-125, 125 μg/mL); cetylpyridinium chloride (CPC) (CPC-50, 50 μg/mL; and CPC-100, 100 μg/mL); CPC loaded MSN (MSN4) (MSN4-50, 50 μg/mL; MSN4-100, 100 μg/mL); or nothing (control) for 16 hr in the presence of 2% sucrose.

FIG. 2B shows the bacterial killing effects on biofilm prepared according to Example III treated with CHX base in solutions (CHX-50, 50 μg/mL; CHX-100, 100 μg/mL); CHX base loaded MSN (MSN3) at various concentrations (MSN3-25, 25 μg/mL; MSN3-50, 50 μg/mL; MSN3-75, 75 μg/mL; MSN3-100, 100 μg/mL; or MSN3-125, 125 μg/mL); cetylpyridinium chloride (CPC) (CPC-50, 50 μg/mL; and CPC-100, 100 μg/mL); CPC loaded MSN (MSN4) (MSN4-50, 50 μg/mL; MSN4-100, 100 μg/mL); or nothing (control) for 16 hr in the presence of 2% sucrose.

FIGS. 3A and 3B show denaturing gradient gel electrophoresis (DGGE) fingerprints from extracted community DNA (microbiome gel fingerprints) of the control and treated microbiomes (bands marked with S show microbiome fingerprints of the treated supernatant, bands marked B show microbiome fingerprints of the treated biofilms, O-Mix refers to the stock model microbiome used for preparation of the biofilm and supernatant for treatment assays).

FIG. 4A shows the pH of media prepared with the model microbiome simulating human oral microbiome according to Example III treated with CHX base solution (100 μg/mL, prepared according to Example I(A)); MSN3 (100 μg/mL and 125 μg/mL of CHX base, prepared according to Example I(B)); or nothing (control).

FIG. 4B shows the optical density (OD at 600 nm) after treatment with CHX base solution (100 μg/mL, prepared according to Example I(A)); MSN3 (100 μg/mL and 125 μg/mL of CHX base, prepared according to Example I(B)); or nothing (control).

FIG. 5 shows an example of a denaturing gradient gel electrophoresis (DGGE) fingerprints obtained from extracted community DNA, which showed that composition of microbiomes of the supernatant and biofilms changed after the CHX base composition treatments, e.g., CHX base solution (100 μg/mL, prepared according to Example I(A)); MSN3 (100 μg/mL and 125 μg/mL of CHX base, prepared according to Example I(B)); or nothing (control). S: supernatant; and B: biofilm.

FIG. 6A shows total colony forming units in supernatant after treatment effects of CHX-G (200 μg/mL), CPC-containing MSN4 (200 μg/mL CPC), and CHX base composition (MSN3, 200 μg/mL of CHX base).

FIG. 6B shows total colony forming units in biofilm after treatment effects of CHX-G (200 μg/mL), CPC-containing MSN4 (200 μg/mL CPC), and CHX base composition (MSN3, 200 μg/mL of CHX base).

FIG. 7 shows the pH of a microbiome treated with MSN3, MSN4, CHX-G or control in the presence with 2% sucrose.

### DETAILED DESCRIPTION

Although biguanide (e.g., CHX) bases have been used as antimicrobial agents, they are often used in one or more salt forms for improved properties, e.g., water solubilities. Therefore, although biguanides (e.g., CHX) are bases, they are often associated with their more commonly used salt forms. As used herein, unless otherwise specified, biguanides (e.g., CHX) mean their base form, and may be also referred to as biguanide (e.g., CHX) bases, to emphasize that compositions and pharmaceutical compositions provided herein uses majorly the base forms of biguanides (e.g., CHX) instead of their more commonly used salt forms.

For examples, as provided in the Example section, CHX base compositions comprising one or more carriers and CHX base as the major CHX form maintained a pH of about 5.5 or higher in a model microbiome simulating human oral microbiome prepared according to Example III with some bacteria survived (Figures IV(B)-1). The one or more carriers may be liquid (e.g., solvent, such as the embodiments prepared according to Example I(A)) and/or solid (e.g., mesoporous silica particles (MSN), such as the embodiments prepared according to Example I(B)). The microbiome tested was a model microbiome simulating human oral microbiome prepared according to Example III, and tested in a form of supernatant (e.g., bands marked “S” in Figures IV(A)-3A & B and IV(B)-3) or a biofilm (e.g., bands marked “B” in Figures IV(A)-3A & B and IV(B)-3).

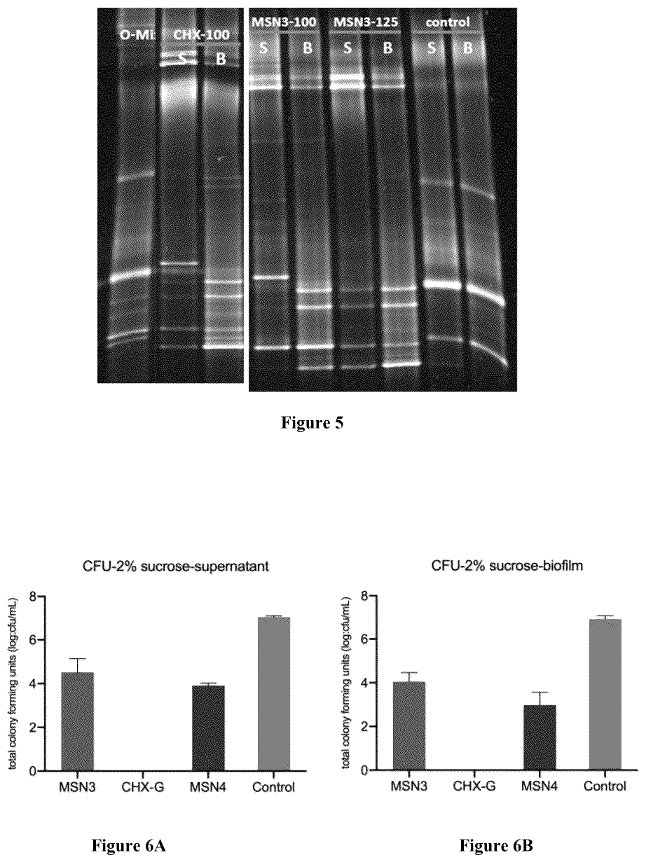

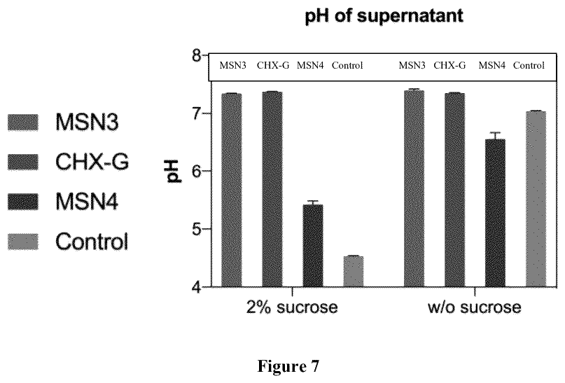

As disclosed in Example V, supernatant and biofilms treated with various embodiments of the CHX base composition had i) a pH above 5.5 while the untreated biofilm had a pH below 5.5 (FIG. 7) and ii) bacteria survived the treatment (FIGS. 6A-6B); while biofilms treated with CHX-G showed a higher pH than the untreated biofilms and no trace of bacteria (FIGS. 6A-6B and 7).

### Biguanide (e.g., CHX) Base Compositions and Pharmaceutical Compositions

One aspect of the invention relates to a biguanide (e.g., CHX) base composition comprising biguanide (e.g., CHX) base, the molar content of biguanide (e.g., CHX) base in all biguanide (e.g., CHX) derivatives comprised in the biguanide (e.g., CHX) base composition being at least about 51%, at least about 60%, at least about 70%, at least about 80%, at least about 90%, at least about 95%, at least about 97%, or at least about 99%.

Examples of biguanides include, without limitation, CHX, metformin, phenformin, buformin, and polyhexanide.

In certain embodiments, the biguanide (e.g., CHX) base composition further comprises one or more carriers. The carriers may be liquid (e.g., solvents), solid (e.g., particles such as porous particles, and may comprise polymers, organic compounds, and/or inorganic substrates such as metal and metal oxide substrates, silica, molecular sieves, carbon, etc.), and/or gels (e.g., hydrogels). Examples of solvents include, without limitation, water, dimethyl sulfoxide, alcohol (e.g., EtOH), and mixtures thereof. Examples for porous particles include, without limitation, mesoporous silica particles (MSN), molecular sieves, and active carbon. The carriers (e.g., particles, substrates) may be surface-functionalized to enhance interactions (e.g., chemical and/or physical interactions) with biguanide (e.g., CHX) base.

In certain embodiments, the one or more carriers are pharmaceutically acceptable carriers, and the biguanide (e.g., CHX) base composition is a biguanide (e.g., CHX) base pharmaceutical composition.

In certain embodiments, the concentrations of biguanide (e.g., CHX) base in the biguanide (e.g., CHX) base composition or pharmaceutical composition is about 50 μg/mL, about 100 μg/mL, about 200 μg/mL, about 300 μg/mL, about 400 μg/mL, about 500 μg/mL, about 1 mg/mL, about 1 mg/mL or lower.

In certain embodiments, the biguanide (e.g., CHX) base composition or pharmaceutical composition is an oral rinse, toothpaste, chewing gum, or hydrogel for mouth guard and mouth tray.

### Uses of the Biguanide (e.g., CHX) Base Compositions and Pharmaceutical Compositions

Another aspect of the invention relates to methods of inhibiting growth of acid-producing bacteria comprising contacting the acid-producing bacteria with an effective amount of the biguanide (e.g., CHX) base composition or a therapeutically effective amount of the biguanide (e.g., CHX) base pharmaceutical composition.

Another aspect of the invention relates to methods of inhibiting growth of acid-producing bacteria in a subject comprising administering to the subject a therapeutically effective amount of the biguanide (e.g., CHX) base pharmaceutical composition.

Another aspect of the invention relates to methods of maintaining a pH of a microbiome at pH 5.5 or higher comprising administering to the microbiome an effective amount of the biguanide (e.g., CHX) base composition or a therapeutically effective amount of the biguanide (e.g., CHX) base pharmaceutical composition, the microbiome comprising acid-producing bacteria and non-acid producing bacteria. In certain embodiments of the method disclosed herein, growth inhibitions of one or more of the acid-producing bacteria in the microbiome are more significantly than those of one or more non-acid-producing bacteria microbiome. In certain embodiments, the biguanide (e.g., CHX) base composition or pharmaceutical composition shows unexpectedly preferred growth inhibition of acid-producing bacteria when compared with treatment of one or more salts of the same biguanide (e.g., CHX) base.

In certain embodiments of the method disclosed herein, the pH of the microbiome was maintained at about 5.5 or higher without eliminating all or substantially all microbiome. In certain embodiments, the administration of the biguanide (e.g., CHX) base composition maintains a healthy pH (pH ≥5.5) while still show high cell-viability, especially for non-acid producing bacteria. In contrast, microbiome treated by biguanide (e.g., CHX) salts, e.g., the gluconate salts of CHX (CHX-G), may keep a neutral pH by effectively killing all bacteria. Preferred growth inhibition of acid-producing bacteria is beneficial in preventing biocorrosion that caused by acidogenic bacteria, while keeping beneficial bacteria, e.g., probiotics.

Another aspect of the invention relates to methods of maintaining a pH of an environment with presence of acid-producing bacteria to about pH 5.5 or higher comprising administering to the environment an effective amount of the biguanide (e.g., CHX) base composition or a therapeutically effective amount of the biguanide (e.g., CHX) base pharmaceutical composition. In certain embodiments, the environment may be an environment in a subject, e.g., oral environment. In certain embodiments, the environment may be an environment in gas pipeline and construction. Certain embodiments of the methods disclosed herein comprise administering an effective amount of the biguanide (e.g., CHX) base composition to a mining field or a drilling field (e.g., oil field) where preferred growth-inhibition or elimination of acid producing bacteria is desired.

Another aspect relates to the use of biguanide (e.g., CHX) base composition and biguanide (e.g., CHX) base pharmaceutical composition for caries prevention in a subject comprising administering to the subject an effective amount of the biguanide (e.g., CHX) base composition or a therapeutically effective amount of the biguanide (e.g., CHX) base pharmaceutical composition.

In certain embodiments of the methods disclosed herein, the biguanide (e.g., CHX) base composition or biguanide (e.g., CHX) base pharmaceutical composition showed preferred growth inhibition of acid-producing bacteria compared to non-acid-producing bacteria.

In certain embodiments of the methods disclosed herein, the effective amount of the biguanide (e.g., CHX) base composition or the pharmaceutically effective amount of the biguanide (e.g., CHX) base pharmaceutical composition may be lower than that of a biguanide (e.g., CHX) salt composition to lower drug accumulation and/or the risk of drug resistance.

In certain embodiments of the methods disclosed herein, the method further avoids tooth staining or other undesired effects of certain salt forms biguanide (e.g., CHX-G for tooth staining).

In certain embodiments of the methods disclosed herein, examples of the acid-producing bacteria include, without limitation, S. mutans.

In certain embodiments of the methods disclosed herein, the effective amount or therapeutically effective amount of biguanide (e.g., CHX) base is below 1 mg/ml

In certain embodiments of the methods disclosed herein, the biguanide (e.g., CHX) base composition or biguanide (e.g., CHX) base pharmaceutical composition is administered once a day, twice a day, three times a day, once every other day, once every three days, once every five days, once every six days, once every week, once every two weeks, once every three weeks, or once a month.

In certain embodiments of the methods disclosed herein, the biguanide (e.g., CHX) base composition or biguanide (e.g., CHX) base pharmaceutical composition is in contact with the acid-producing bacteria for a first contact time, e.g., 10 seconds, 30 seconds, 1 min, 5 min, 10 min, 30 min, 1 hr, 2 hrs, 5 hrs, 10 hrs, 15 hrs, 16 hrs, 1 day, 2 days, 3 days, 4 days, 5 days, 6 days, 1 week, 2 weeks, 3 weeks, 4 weeks, 1 month, 2 months, 3 months, 4 months, 5 months, 6 months, 1 year, or longer.

### Preparations of Biguanide (e.g., CHX) Base Composition and Pharmaceutical Biguanide (e.g., CHX) Base Composition

Another aspect of the invention relates to preparation of the biguanide (e.g., CHX) base composition or biguanide (e.g., CHX) base pharmaceutical composition, comprising loading the biguanide (e.g., CHX) base for action with one or more carriers or pharmaceutically acceptable carriers by physical interactions, chemical interactions or combinations thereof. Examples for physical interactions include, without limitation, intermolecular forces, absorptions, and capillary effects. Examples of chemical interactions include, without limitation, hydrogen bonding, acid-base interactions, and proton exchanges.

The term “about” is used herein to refer to a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

The terms “treat,” “treating,” or “treatment” are used herein to refer to ameliorating a disease or disorder (i.e., slowing or arresting or reducing the development of the disease or at least one of the clinical symptoms thereof). The terms also refer to alleviating or ameliorating at least one physical parameter including those which may not be discernible by the patient. The terms also refer to modulating the disease or disorder, either physically (e.g., through stabilization of a discernible symptom), physiologically, (e.g., through stabilization of a physical parameter), or both. The terms also refer to preventing or delaying the onset or development or progression of the disease or disorder.

The term “therapeutically effective amount” is used herein to refer to the amount of a therapeutic agent or composition effective in prevention or treatment of a disorder or disease.

The term “pharmaceutically acceptable” is used herein to refer to a molecular entity or composition that is pharmaceutically useful and not biologically or otherwise undesirable.

The term “carrier” is used herein to refer to a diluent, adjuvant, excipient, or vehicle with which the compound is administered.

The term “excipient” as used herein refers to any ingredient in a pharmaceutical composition other than the active ingredient.

Unless otherwise defined, all other scientific and technical terms have the same meaning as commonly understood to one of ordinary skill in the art. Such scientific and technical terms are explained in the literature, for example: J. Sambrook, E. F. Fritsch, and T Maniatis, 1989, Molecular Cloning: A Laboratory Manual, Second Edition, Books 1-3, Cold Spring Harbor Laboratory Press; Martin, 1990, Remington's Pharmaceutical Sciences, 18th Edition, Mack Publishing Co; Glover, 1985, DNA Cloning: A Practical Approach, Volumes I and II, MRL Press, Ltd.; and Ausubel, F., Brent, R., Kingston, R. E., Moore, D. D., Seidman, J. G., Smith, J. A., Struhl, K., 2002, Current Protocols in Molecular Biology, Greene Publishing Associates/Wiley Intersciences.

The following examples are intended to illustrate various embodiments of the invention. As such, the specific embodiments discussed are not to be construed as limitations on the scope of the invention. It will be apparent to one skilled int eh art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example I. Preparation of Embodiments of CHX Base Composition and CHX Base Pharmaceutical Composition

### I(A) Preparation of Solution Embodiments of the CHX Base Composition

A stock solution of CHX base in dimethyl sulfoxide at a concentration of 10 mg/mL was prepared. This stock solution was diluted into solution embodiments at various concentrations, e.g., 50 μg/mL, 100 μg/mL . . . and 500 μg/mL. For CHX base pharmaceutical composition, the concentration of dimethyl sulfoxide was kept below 5 v %. These solution embodiments of CHX base composition and CHX base pharmaceutical composition were used to treat model microbiome simulating human oral microbiome prepared according to Example III in Examples IV and V.

### I(B) Preparation of Embodiments of CHX Base Composition and CHX Base Pharmaceutical Composition Comprising Porous Particles

### I(B)(i) Preparation of Embodiments of CHX Base Composition Comprising Unfunctionalized Mesoporous Silica Particles (MSN)

CHX base was dissolve in ethanol to obtain a CHX base-EtOH solution with CHX base concentration of 10 mg/mL. Mesoporous silica particles (MSN, 200 nm in diameter and 4 nm pore size) was added to the CHX base-EtOH solution and agitated overnight. The obtained mixture was then centrifuged at 3,000 rpm for 10 min to collect the CHX base-loaded MSN, which was dried overnight at 50° C. and 1 mmHg. The CHX base-loaded MSN obtained contained (23±2) wt % of CHX base determined by thermalgravimetric analysis and CHX base release.

### I(B)(ii) Preparation of Embodiments of CHX Base Composition (MSN3) Comprising Surface Functionalized MSN

The surface functionalized MSN used to prepare MSN3 was obtained by a two-step functionalization of the MSsaline.

N used in Example I(B)(i): 1) silanization with alkyl bromine and 2) Menschutkin reaction with (E)-4-(pyridin-4-yldiazenyl) phenyl isobutyrate. Pre-hydrolyzed 7-bromoheptyltrimethoxysilane (200 μL) was stirred with 20 mL of ethanol at room temperature for 2 h. Then 500 mg of MSN in ethanol was added and stirred for 2 h at 50° C. The mixture obtained was then centrifuged to provide the alkyl-bromine silanized MSN, which was washed with ethanol three times and dried in vacuum overnight. Then the functionalization was finalized by attaching (E)-4-(pyridin-4-yldiazenyl)phenyl isobutyrate through Menschutkin reaction at 80° C. for 3 days (M B Smith, J March. March's Advanced Organic Chemistry (Wiley, 2001) (ISBN 0-471-58589-0)). The attached surface functional group of MSN3 was acidic and had a pKa of around 5.3. The acid-base interactions between the CHX base and the acidic surface function group of MSN3 may be affected by environmental pH and/or the presence of other acid(s) and/or base(s).

The CHX base was loaded to MSN3 by mixing the CHX base (5 mg/mL) and the functionalized MSN together in chloroform and stirred for 12 h at room temperature. The product was collected through centrifugation at 3,000 rpm for 5 min and washed five times using chloroform. After dried in vacuum at 50° C. overnight, thermalgravimetric analysis and CHX base release were used to determine the concentration of CHX base in the CHX base composition MSN3, which was (25±2) wt %.

### I(B)(iii) Preparation of Embodiments of Cetylpyridinium Chloride (CPC) Composition (MSN4) Comprising Surface Functionalized MSN Disclosed in Example I(B)(ii)

CPC was an antiseptic drug. CPC (10 mg/mL) was mixed and stirred with the surface functionalized MSN disclosed in Example I(B)(ii) for 12 h at room temperature. The product obtained was collected by centrifugation and dried in vacuum at 50° C. overnight. No washing was performed. Thermalgravimetric analysis and CPC release were used to determine the concentration of CPC in the CPC containing MSN4.

### Example II. CHX Base Release Performance of MSN3 and CPC Release Performance of MSN4

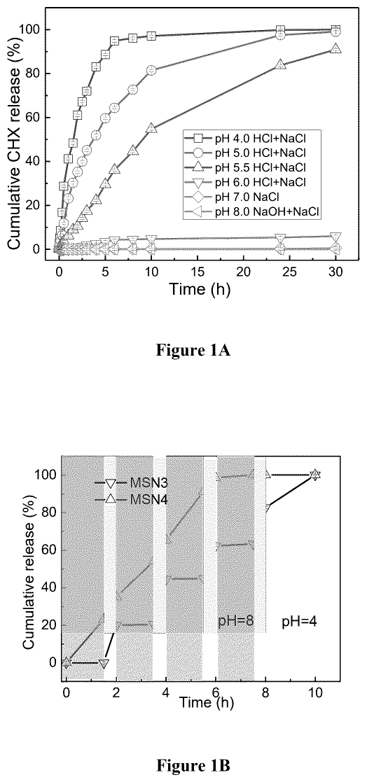

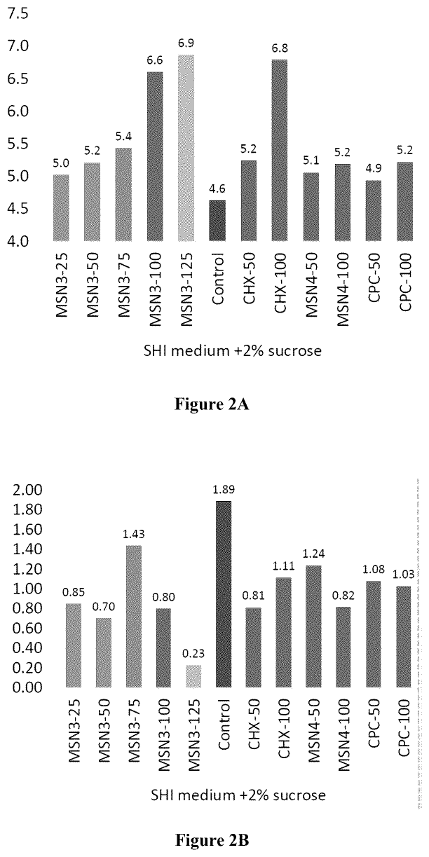

The drug release from ssMSN3 and MSN4 (2 mg each) was carried out using a dialysis tubing (12,000-14,000 Dalton) dialyzed in 20 mL buffers of various pH values under constant stirring at 37° C. At predetermined time intervals, 1 mL aliquots were taken for UV-vis analysis to determine CHX base concentration and CPC concentration at 254 nm and 259 nm, respectively. MSN3 had a pH-responsive CHX release (FIG. 1A), specifically, CHX base was released faster in buffer with pH below 5.5. There was no CHX base released at pH above 7. In contrast, CPC was released at the same rate in different buffers with different pH (FIG. 2B). Switching between buffers at pH 4 and pH 8, release of CHX from MSN3 was on and off while CPC was release continuously at the same rate (FIG. 2B).

### Example III. Preparation of a Model Microbiome Simulating Human Oral Microbiome, Saliva-Derived Multispecies Biofilms Prepared Using Same, and Characterizations of Same

Saliva samples were collected and pooled from 5 healthy volunteers as a model microbiome simulating human oral microbiome, and used to inoculate SHI medium containing different test compositions and controls.

1 mL pooled saliva sample was added to each well of a 24-well plate that had been pre-coated with sterilized, cell-free saliva. The plate was incubated at 37° C. for 16 h in microaerobic conditions (2% O₂, 5% CO₂, balanced with nitrogen) to allow biofilm formation. After incubation, planktonic portion was collected, optical density was measured at 600 nm and cells were pelleted for DNA isolation. Meanwhile, 1 mL of SHI medium was added to the biofilm portion of each well, biofilm cells were dispersed by scraping and vortex to break the cell aggregates. Optical density was measured at 600 nm and cells were collected for DNA isolation.

PCR-DGGE analysis. Total genomic DNA of bacterial samples was isolated using the MasterPure™ DNA purification kit (Epicentre). DNA quality and quantity were determined by a Nanodrop 2000 Spectrophotometer. Amplification of bacterial 16S rRNA genes by PCR was carried out. Briefly, the universal primer set, Bac1 (5′-CGCCCGCCGCGCCCCGCGCCCGTCCCGCCGCCCCCGCCCGACTACGTGCCAGCAGCC-3′) and Bac2 (5′-GGACTACCAGGGTATCTAATCC-3′), were used to amply an approximately 300-bp internal fragment of the 16S rRNA gene. Each 50-μl PCR reaction contained 100 ng of purified genomic DNA, 40 pmol of each primer, 200 μM of each dNTP, 4.0 mM MgCl₂, 5 μl of 10× PCR buffer, and 2.5 U of Taq DNA polymerase (Invitrogen). Cycling conditions were 94° C. for 3 min, followed by 30 cycles of 94° C. for 1 min, 56° C. for 1 min and 72° C. for 30 s, with a final extension period of 5 min at 72° C. The resulting PCR products were evaluated by electrophoresis in 1.0% agarose gels.

Polyacrylamide gels at an 8% concentration were prepared with a denaturing urea/formamide gradient between 40% (containing 2.8 M urea and 16% (v/v) formamide) and 70% (containing 4.9 M urea and 28% (v/v) formamide). Approximately 300 ng of the PCR product were applied per lane. The gels were submerged in 1×TAE (Tris-Acetate-EDTA) buffer (40 mM Tris base, 40 mM glacial acid acetic, 1 mM EDTA) and the PCR products were separated by electrophoresis for 17 h at 58° C. using a fixed voltage of 60 V in the Bio-Rad DCode System (Bio-Rad laboratories, Inc. Hercules, CA, USA). After electrophoresis, the gels were rinsed and stained for 15 min in 1×TAE buffer containing 0.5 μg/ml ethidium bromide, followed by 10 min of de-staining in 1×TAE buffer. DGGE profile images were digitally recorded using the Molecular Imager Gel Documentation system (BioRad). Diversity Database Software (BioRad) was used to assess the change in the relative intensity of bands corresponding to bacterial species of interest.

Identification of bacterial species in DGGE gel. Bands of interest were excised from the DGGE gels and transferred to a 1.5 ml microfuge tube containing 10 μl of sterile ddH2O. Tubes were incubated at 4° C. overnight before the recovered DNA samples were reamplified with the universal primer set (Bac1 and Bac2). The PCR products were purified using the QIAquick PCR purification kit (Qiagen) and sequenced at the UCLA Sequencing and Genotyping Core Facility. The obtained partial 16S rRNA gene sequences (about 300 bp) were used to BLAST search against the HOMD (http://www.homd.org) and NCBI (http://www.ncbi.nlm.nih.gov) databases. Sequences with 98-100% identity to those deposited in the public domain databases were considered to be positive identification of taxa.

### Example IV. Preferred Growth Inhibition of Acid-Producing Bacteria by Various Embodiments of the CHX Base Composition (Solutions and MSN3)

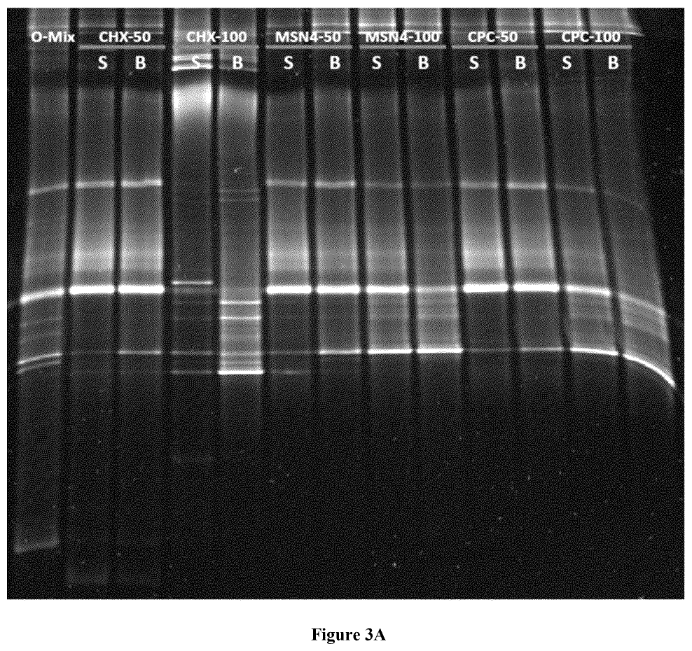

Supernatant and biofilms prepared with the model microbiome simulating human oral microbiome according to Example III were treated with embodiments of the CHX base composition (e.g., solution embodiments of the CHX base composition prepared according to Example I (the CHX base solutions); and MSN3 prepared according to Example II(B)(ii)). CHX base treatment increased the pH of the microbiome up to pH 6.9 without complete elimination of bacteria (FIGS. 2A-2B & 3A-3B). The higher CHX base concentration, the higher pH of the treated microbiome (FIGS. 2A-2B). Although CPC treatment also increased the pH of the microbiome (FIGS. 2A-2B & 3A-3B), increase of CPC concentration did not raise the pH of the treated microbiome to above the healthy pH 5.5 (FIG. 2A). Furthermore, CHX base compositions with certain different CHX base concentrations also showed different microbiome gel fingerprints (FIGS. 3A-3B).

IV(A): Effects on Microbiomes with Various Embodiments of CHX Base Compositions with Various CHX Base Concentrations and CPC Compositions with Various CPC Concentrations

Supernatant and biofilm prepared according to Example III was treated with CHX base in solutions (CHX-50, 50 μg/mL; CHX-100, 100 μg/mL); CHX base loaded MSN (MSN3) at various concentrations (MSN3-25, 25 μg/mL; MSN3-50, 50 μg/mL; MSN3-75, 75 μg/mL; MSN3-100, 100 μg/mL; or MSN3-125, 125 μg/mL); cetylpyridinium chloride (CPC) (CPC-50, 50 μg/mL; and CPC-100, 100 μg/mL); CPC loaded MSN (MSN4) (MSN4-50, 50 μg/mL; MSN4-100, 100 μg/mL); or nothing (control) for 16 hr in the presence of 2% sucrose (FIGS. 2A-2B). Biofilms treated with CHX base in solution concentration or CHX base MSN compositions had a higher pH than the control biofilm (FIG. 2A). However, the pH of the treated biofilm appeared to depend on the CHX base concentration administered. The pH of the biofilm treated with CHX base solutions and CHX base loaded MSN remained under 5.5 when the CHX base concentration was 75 μg/mL or lower. While the pH of the biofilm treated with CPC compositions remained lower than 5.5 for CPC concentrations of 50 and 100 μg/mL, the pH of the biofilm treated with CHX base concentration of 100 and 125 μg/mL exceeded 5.5.

FIGS. 3A & 3B show denaturing gradient gel electrophoresis (DGGE) fingerprints from extracted community DNA (microbiome gel fingerprints) of the control and treated microbiomes (bands marked with S show microbiome fingerprints of the treated supernatant, bands marked B show microbiome fingerprints of the treated biofilms, O-Mix refers to the stock model microbiome used for preparation of the biofilm and supernatant for treatment assays).

More experiments may be carried out to further characterize the species of the microbiome treated with various compositions.

IV(B): Effects on Microbiomes with Embodiments of CHX Base Compositions with CHX Base Concentrations of 100 μg/mL and Higher

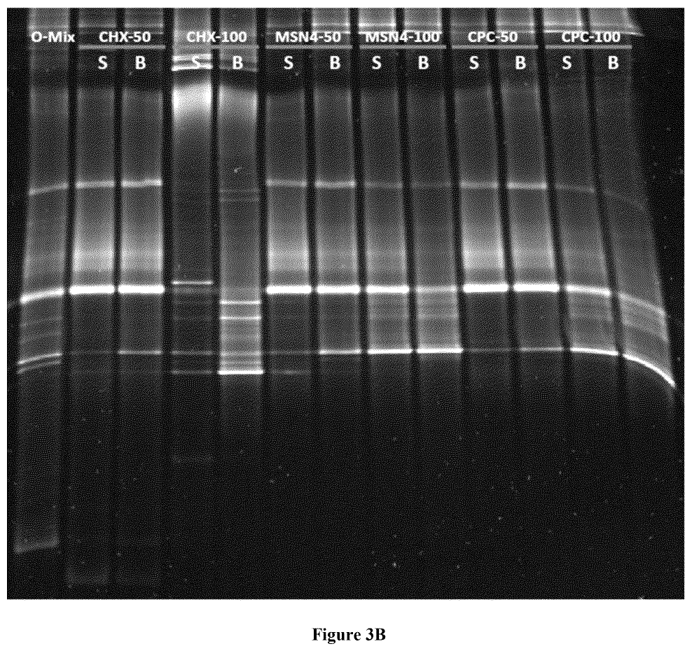

Supernatant and biofilms prepared with the model microbiome simulating human oral microbiome according to Example III were treated with CHX base solution (100 μg/mL, prepared according to Example I(A)); MSN3 (100 μg/mL and 125 μg/mL of CHX base, prepared according to Example I(B)); or nothing (control). Microbiomes treated with CHX base compositions showed abundant of cells and pH >5.5 while the untreated microbiomes had a pH of 4.6 (FIG. 4A).

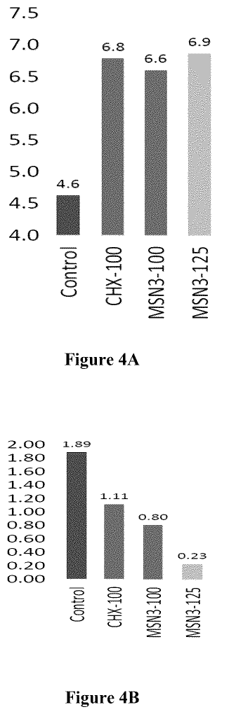

Denaturing gradient gel electrophoresis (DGGE) fingerprints from extracted community DNA were obtained, and showed that composition of microbiomes of the supernatant and biofilms changed after the CHX base composition treatments (FIG. 5, S: supernatant; and B: biofilm). In addition, the pH and bacteria composition after the CHX base composition treatment appeared to be impacted by the different CHX base concentrations, time of treatment, and the carriers the embodiments of the CHX base composition comprised. For example, MSN3 with higher CHX base concentration (125 μg/mL, MSN3-125) killed more bacteria than MSN3 with lower CHX base concentration (100 μg/mL, MSN3-100); and treatment with CHX base compositions having the same CHX base concentration (100 μg/mL) but different carriers showed different bacterial killing effects (FIG. 4B).

### Example V. CHX-G and CPC Killed More Bacteria than Embodiments of CHX Base Composition

CHX-G and CPC were also evaluated in the same model microbiome prepared in supernatant and biofilms according to Example III. However, both CHX-G (200 μg/mL) and CPC containing MSN4 (200 μg/mL of CPC) killed more bacteria than an embodiment of CHX base composition (MSN3, 200 μg/mL of CHX base) (FIGS. 6A-6B).

The microbiome treated with MSN3 in the presence with 2% sucrose produced much less acid and had a pH >5.5 while the microbiome treated with MSN4 had a pH <5.5, which suggested less effective growth inhibit of acid-producing bacteria by MSN4 (FIG. 7).

### Example VI. Use CHX Base Composition in Oral Rinse

Embodiments of the CHX base composition at an effective concentration may be added in oral rinse as an active component replacing CHX salt. The oral rinse with CHX base composition may be used for preventing dental caries and other oral disease without side effects from the CHX salts, e.g., tooth staining caused by CHX-G.

### Example VII. Use CHX Base Composition in Chewing Gum

Embodiments of the CHX base composition at an effective concentration may be added in chewing gum as an active antimicrobial component. The chewing gum with CHX base composition may be used for preventing dental caries and other oral disease without side effects from the CHX salts, e.g., tooth staining caused by CHX-G.

### Example VIII. Use CHX Base Composition in Hydrogels for Mouth Guards or Mouth Trays

Embodiments of the CHX base composition at an effective concentration may be added in hydrogels for mouth guards or mouth trays. The hydrogel and/or devices with CHX base composition may be used for preventing dental caries and other oral disease without side effects from the CHX salts, e.g., tooth staining caused by CHX-G.

### Example IX. Use CHX Base Composition for Preventing Biocorrosion in Oil Pipelines and Constructions

In the case of industrial bio-corrosion, where certain microbial metabolic activities promote deterioration of the underlying metal structures, it is often a group of sulfate reducing, or acid-producing bacteria that contribute to the process. Embodiments of the CHX base composition disclosed herein can be added in pipelines or construction materials through coating with carries. Such pipelines and construction materials with CHX base composition may inhibit the growth of acid-producing bacteria, consequently, reduce maintenance cost and improve service life of these materials.

1-21. (canceled)

22. A composition comprising one or more carriers and a biguanide majorly in its base form, wherein the biguanide is selected from the group consisting of CHX, metformin, phenformin, buformin, and polyhexanide; and the biguanide base is selected from the group consisting of CHX base, metformin base, phenformin base, buformin base, and polyhexanide base.

23. The composition of claim 22, being a pharmaceutical composition, wherein the one or more carriers are pharmaceutically acceptable carriers.

24. The composition of claim 22, wherein the molar content of the biguanide base in all biguanide derivatives comprised in the composition or the pharmaceutical composition is at least about 51%, at least about 60%, at least about 70%, at least about 80%, at least about 90%, at least about 95%, at least about 97%, or at least about 99%.

25. The composition of claim 23, wherein the molar content of the biguanide base in all biguanide derivatives comprised in the composition or the pharmaceutical composition is at least about 51%, at least about 60%, at least about 70%, at least about 80%, at least about 90%, at least about 95%, at least about 97%, or at least about 99%.

26. The composition of claim 22, wherein the one or more carriers are liquid comprising one or more solvents selected from the group consisting of water, dimethyl sulfoxide, alcohols, and mixtures thereof.

27. The composition of claim 23, wherein the one or more pharmaceutically acceptable carriers are liquid comprising one or more solvents selected from the group consisting of water, dimethyl sulfoxide, alcohols, and mixtures thereof.

28. The composition of claim 22, wherein the concentration of the biguanide base in the composition is about 50 μg/mL, about 100 μg/mL, about 200 μg/mL, about 300 μg/mL, about 400 μg/mL, about 500 μg/mL, about 1 mg/mL, about 1 mg/mL or lower.

29. The composition of claim 22, wherein the composition is an oral rinse, toothpaste, chewing gum, or hydrogel for mouth guard and mouth tray.

30. A method of inhibiting growth of acid-producing bacteria comprising contacting the acid-producing bacteria with an effective amount of the composition of claim 22.

31. A method of inhibiting growth of acid-producing bacteria comprising contacting the acid-producing bacteria with a therapeutically effective amount of the composition of claim 23.

32. A method of inhibiting growth of acid-producing bacteria in a subject comprising administering to the subject an effective amount of the composition of claim 22.

33. A method of inhibiting growth of acid-producing bacteria in a subject comprising administering to the subject a therapeutically effective amount of the composition of claim 23.

34. A method of maintaining a pH of a microbiome at pH 5.5 or higher comprising administering to the microbiome an effective amount of the composition of claim 22, the microbiome comprising acid-producing bacteria and non-acid producing bacteria.

35. A method of maintaining a pH of a microbiome at pH 5.5 or higher comprising administering to the microbiome a therapeutically effective amount of the composition of claim 23, the microbiome comprising acid-producing bacteria and non-acid producing bacteria.

36. A method of maintaining a pH of an environment with presence of acid-producing bacteria to about pH 5.5 or higher comprising administering to the environment an effective amount of the composition of claim 22.

37. A method of maintaining a pH of an environment with presence of acid-producing bacteria to about pH 5.5 or higher comprising administering to the environment a therapeutically effective amount of the composition of claim 23.

38. The method of claim 32, growth inhibitions of one or more of the acid-producing bacteria in the microbiome are more significantly than those of one or more non-acid-producing bacteria microbiome.

39. The method of claim 33, growth inhibitions of one or more of the acid-producing bacteria in the microbiome are more significantly than those of one or more non-acid-producing bacteria microbiome.

40. A method of treating or preventing caries in a subject comprising administering to the subject an effective amount of the composition of claim 22.

41. A method of treating or preventing caries in a subject comprising administering to the subject an effective amount of the composition of claim 23.
